(19) United States
(12) Patent Application Publication
(10) Pub. No.: US 2009/0250603 A1
(43) Pub. Date: Oct. 8, 2009

(54) MAGNETIC ANALYZER APPARATUS AND METHOD FOR ION IMPLANTATION

(76) Inventor: Hilton F. Glavish, Incline Village, NV (US)

Correspondence Address: KATTEN MUCHIN ROSENMAN LLP;(C/O PATENT ADMINISTRATOR), 2900 K STREET NW, SUITE 200, WASHINGTON, DC, 20007-5118, US

(21) Appl. No.: 12/303,485

(22) PCT Filed: Jun. 13, 2007
(86) PCT No.: PCT/US07/13850

§ 371 Date: Dec. 4, 2008

Related U.S. Application Data
(60) Provisional application No. 60/804,639, filed on Jun. 13, 2006.

(51) Int. Cl. H01J 49/00; A61N 5/00

(52) U.S. Cl. 250/281; 250/492.3

(57) ABSTRACT

In a magnetic analysis apparatus, high voltage insulation (86, 94) isolates the magnet excitation coil (40), power leads (90) and cooling fluid lines (92) from the ferromagnetic assembly (26, 28, 30, 32, 34) of a sector magnet, and the coil supply is disposed in a grounded housing (E). A sleeve (94), containing electric power leads and cooling fluid lines, forms an insulator through the magnet assembly to the coil (40) and the coil is surrounded by electrical insulation providing electrical isolation from the magnet assembly of least 20 KV. The excitation coil comprises alternating coil segments (80) and cooling plates (82) within an impervious cocoon (86) of insulating material of at least 6 mm thickness. Yoke and core members (20, 30, 32, 34) of the magnet assembly are disposed outside of the vacuum housing (20) while pole members (28) extend through and are sealed to walls of the vacuum housing. An ion decelerator (60, 61, 62) is in a housing extension at the same voltage potential as the mass analyzer housing.

### BACKGROUND

The present invention relates to ion implanting into semiconductor wafers, and more particularly to magnetic analyzer configurations useful for decelerating ion beams after magnetic analysis.

In commercial ion implanters the ions extracted from an ion source are typically formed into a beam and passed through a sector type dipole magnet in order to select a specific ion species before the beam is irradiated on a semiconductor wafer. At implantation energies below 10-20 keV the ions are decelerated after magnetic analysis. Generally, this procedure produces higher beam currents on the wafer compared with the direct approach of simply extracting the ions at a low energy from the ion source prior to magnetic analysis. This is because the internal space charge forces and the intrinsic thermal temperature of an ion beam limit the number of ions that can be extracted from a source and transported through a magnetic analyzer at a low energy. The higher beam currents enable faster ion implantation and more efficient use of capital equipment.

### SUMMARY

A drawback of using post analysis deceleration is that the magnetic analyzer and associated vacuum housing through which the beam is transported as it passes through the analyzer magnet must be high voltage isolated from ground potential, or alternatively the associated vacuum housing must be high voltage isolated from the magnet body. Generally this is inconvenient and costly to implement in practice and in some cases can be limiting for a system. I have realized that an analyzer magnet system that enables the necessary electrical isolation to be achieved conveniently, at low cost, and without loss of magnetic efficiency, can be attained by electronically isolating the coil itself from an analyzer magnet at high voltage. This has the advantage that the magnet coil power supply and cooling fluid system can be kept at ground potential even when ion deceleration is active. It has particular advantage in large systems, i.e. in which the magnet consumes in excess of about 20 KW.

According to one aspect of invention, a magnetic analysis apparatus is provided for use with a decelerator for post analysis deceleration of ions for ion implantation, the apparatus comprising a sector magnet associated with a vacuum housing of nonmagnetic material through which an ion beam passes, the sector magnet having a magnet assembly of ferromagnetic material defining a magnetic field gap to which the ion beam is exposed for mass separation and an excitation coil closely associated with the magnet assembly, the coil connected to power leads extending to a power supply and cooling fluid lines extending to a cooling fluid source and drain, wherein high voltage insulation isolates the closely associated excitation coil, power leads and cooling fluid lines from the magnet assembly and the power supply is disposed in a grounded housing.

Preferred embodiments feature one or more of the following features.

The analyzer magnet and the power supply are constructed to operate with power of at least 20 kilowatts.

At least one sleeve forming a high voltage insulator extends through a portion of the magnet assembly to the excitation coil, the sleeve containing the electrical power leads and cooling fluid lines.

The excitation coil is surrounded by electrical insulation capable of providing electrical isolation from the magnet assembly of least 20 kV.

The excitation coil comprises an assembly of alternating coil segments and cooling plates having coolant passages, the excitation coil connected to the power leads and the cooling plates connected to the cooling fluid lines, and a high voltage insulator layer encapsulates the assembly, preferably the high voltage insulator layer being in the form of an impervious cocoon of insulating material of at least 6 mm thickness.

The apparatus is associated with a vacuum housing held at the same voltage potential as the magnet assembly, the magnet assembly comprising yoke and core members disposed outside of the housing and pole members that extend through and are sealed to walls of the vacuum housing, faces of the pole members at the inside of the housing defining the gap for the ion beam and surfaces of the pole members at the outside of the housing defining flux interfaces removably related to matching surfaces of the core members of the magnet assembly.

The vacuum housing for the mass analyzer has a housing extension in which an ion decelerator is mounted, the housing extension constructed to be held at the same voltage potential as the housing of the mass analyzer. Preferably the decelerator comprises an assembly that includes a final energy electrode, the final energy electrode supported from the housing for the mass analyzer by a high voltage insulator.

The mass analyzer is enclosed in a high voltage enclosure that is isolated by high voltage insulators from electrical ground, and the power supply for the excitation coil is outside of the high voltage enclosure.

The cooling fluid supply line is connected to a source of water not de-ionized.

The sector magnet extends over an arc of about 120 degrees and defines a gap of at least 100 mm dimension.

Another aspect of invention comprises conducting ion implantation implemented by use of the apparatus of any of the foregoing features.

### BRIEF DESCRIPTION OF THE DRAWINGS

FIG. 1 is a schematic plan view of an ion implanter employing a sector type dipole magnetic analyzer followed by an ion decelerator.

FIG. 2 is a cross-sectional view taken through the magnetic analyzer of FIG. 1 along section lines A-A and B-B.

FIG. 3. is an enlarged view of the decelerator shown in FIG. 1.

FIG. 4. is an enlarged cross-section of the high voltage isolated coil shown in FIG. 2.

FIG. 5. shows further details of the coil in FIG. 4.

### DETAILED DESCRIPTION OF EMBODIMENTS OF THE INVENTION

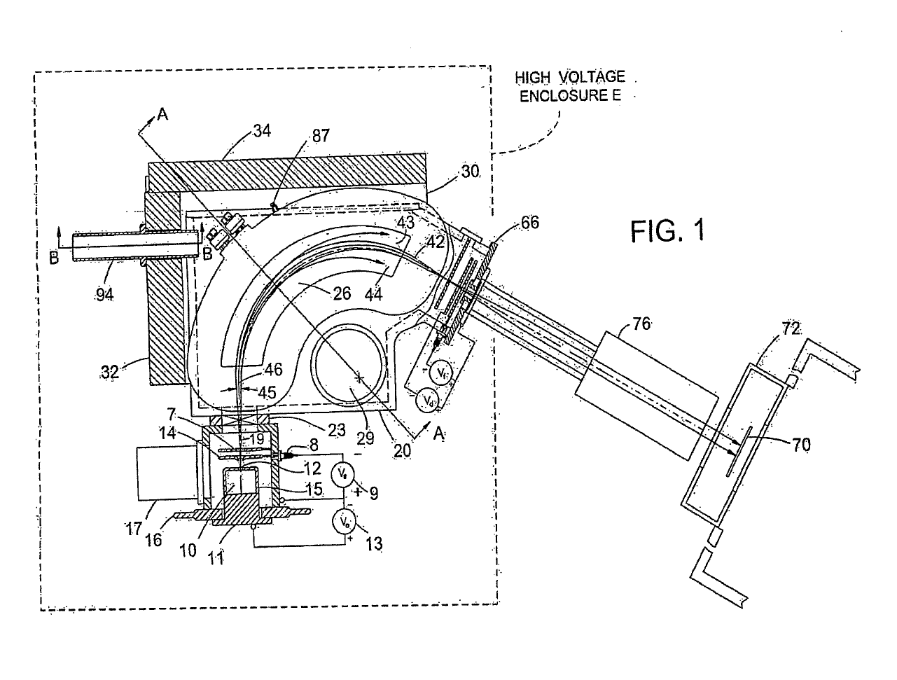

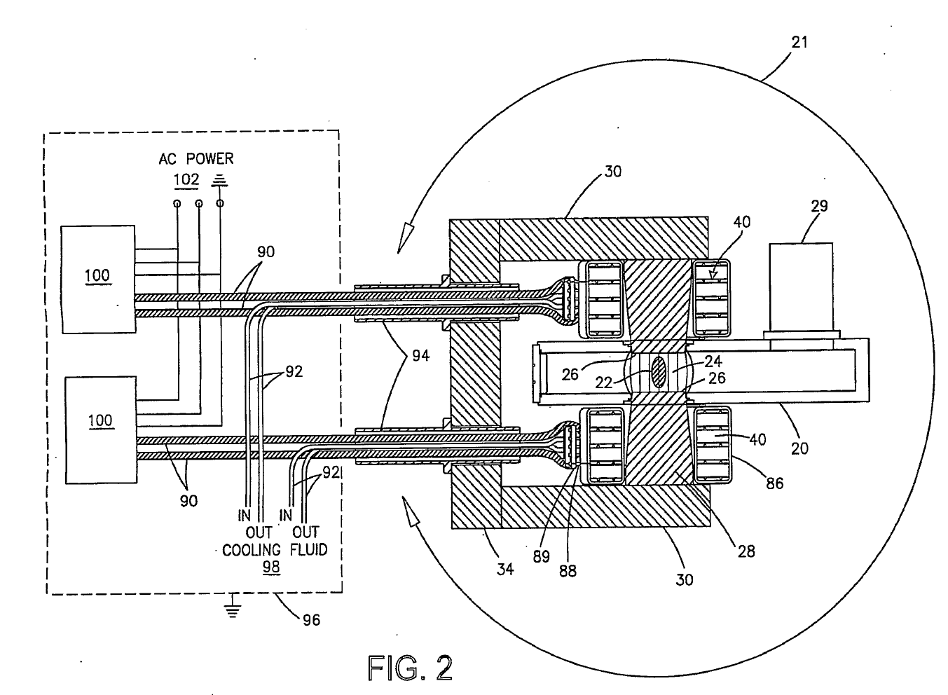

Referring now to the drawings, wherein identical parts are referenced by identical reference numerals, FIGS. 1 and 2 schematically illustrate an ion implanter using post analysis deceleration.

Ions are extracted from an ion source chamber 10 inside an ion source body 11 through an aperture 12 by an accelerating electric voltage (Vₑ) 13, typically in the range of 1 kV to 80 kV, applied between an extraction electrode 14 and the ion source chamber 10. Back-streaming electrons are suppressed by applying to extraction electrode 14 a voltage (Vₛ) 9, of 2-10 kV negative with respect to the ion source vacuum housing 15 and suppressor electrode 7 via an insulated feed-through 8. The suppressor electrode 7 is at the same potential as the ion source vacuum housing 15. The ion source body 10 is insulated from the ion source vacuum housing 15 by an insulator 16. The aperture is 12 is often slot shaped but can also be circular or elliptical. For slot shaped apertures typical dimensions are 3-15 mm wide by 40-150 mm high. A vacuum of typically between about 10⁻⁶ and 10⁻⁴ torr is maintained in the ion source vacuum housing by a vacuum pump 17. The electric field generated between the extraction electrode 14 and the ion source body 11 and aperture 12 forms an approximately mono-energetic beam of ions 19 with dimensions similar to those of the extraction aperture 12.

The beam 19 then passes into the magnet vacuum housing 20 wherein it enters the magnetic field gap of the sector dipole magnet 21, comprising in addition to the vacuum housing, ferromagnetic poles 26, cores 28, yoke cheeks 30, and yoke returns 32 and 34. Referring, in particular to FIG. 2, passing electric current through the coil assemblies 40 generates a magnetic field 24 generally in the vertical direction in the gap between the poles 26. “Vertical” is defined as the direction normal to the generally “horizontal” bending plane of the magnetic analyzer. A vacuum of typically between about 10⁻⁶ and 3×10⁻⁵ torr is maintained in vacuum housing 20 by vacuum pump 29. In order to facilitate maintenance ease of the ion source 10, 11, the ion source housing 15 is isolatable from the magnet vacuum housing 20 with a vacuum valve 23. The magnet housing 20 is of non-ferromagnetic material to prevent interaction with the magnet.

The radial force generated by the magnetic field 24 acting on the electrical charge of the ions, causes the ions to describe substantially circular paths 42, 43, and 44 in the horizontal bending plane of the magnet 21. Since the ions extracted from the ion source chamber 10 all have approximately the same energy, magnet 21 spatially separates the trajectories of ions 43 and 44 possessing respectively higher and lower mass than the desired ions 42 as shown in FIG. 1. The gap space between the poles 26 is typically 30 to 150 mm and the magnitude of the magnetic field 24 ranges from less than one kilo-Gauss to 15 kilo-Gauss. For these parameters, the circular path for desired ions 42 typically has a radius of 200-1000 mm. The beam of desired ions 42 occupies a cross-section 22 approximately as shown in FIG. 2.

Referring to FIGS. 1 and 2, the ion paths entering the magnetic field generally have a range of angles 45 with respect to the central reference path 46. In one embodiment the shape of the pole 26 generates a magnetic field 24 in the gap that causes the ion paths to re-converge at the exit of the magnet and become focused through a mass resolving aperture formed in a blocking plate 51 at a position along the beam path which is ion optically a conjugate image point of the ion source aperture 12 for horizontal ion motion. This enables the horizontal width of the aperture 50 to be minimized and become comparable in dimension to the horizontal aperture width of the ion source aperture 12, without blocking ions of a desired mass. The unwanted ions 43, 44 are stopped by the plate 51. The well known art of designing poles 26 to have this focusing property is described in detail by Enge, Focusing of Charged Particles, Chapter 4.2 Deflecting Magnets, Ed. A. Septier, pp. 203-264. This embodiment is well suited to slot shaped source apertures wherein the long dimension of the slot is oriented in the vertical direction.

In another embodiment, as described for example by White et al, U.S. Pat. No. 5,350,926, the long dimension of the slot is oriented horizontally. In this case the source aperture 12 and extraction electrode 14 are shaped to cause the ions to be focused into the aperture 50 and thus provide effective mass selection even though the aperture 50 is not a conjugate image of the long dimension of the source aperture slot.

An important aspect of the embodiment shown in FIG. 2 is that the poles 26 penetrate through and seal into the vacuum housing 20, an arrangement, which, in effect, maximizes the magnetic efficiency because the space between the poles 26 is not reduced by the presence of the non-ferromagnetic material typically used for the construction of the vacuum housing. The magnetic efficiency is further improved because there is no air gap between the adjacent surfaces of the poles 26 and cores 28. The vacuum housing 20 and poles 26 are sandwiched between the surfaces of the cores 28 but can be easily withdrawn without disassembling the other parts of the magnet, which, in effect, minimizes the cost of maintenance.

As suggested in FIG. 1, the magnet 21 and other high voltage components of the system are typically enclosed within a high voltage safety enclosure isolated by high voltage insulators from the ground.

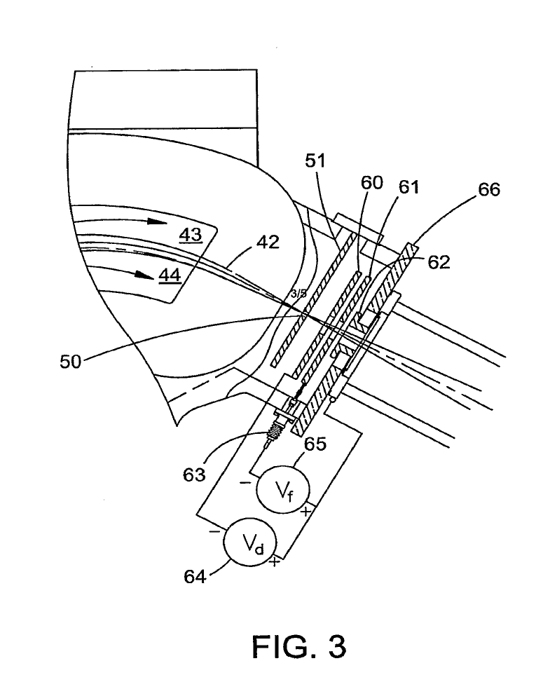

Following mass analysis via the mass resolving aperture 50 and the blocking plate 51, the beam passes through a sequence of three non-ferromagnetic electrodes 60, 61, and 62, as shown in FIGS. 1 and 3. A decelerating voltage (V_{d}) 64, typically 0-30 kV in magnitude, can be applied between electrodes 60 and 62 to decelerate ions to a lower energy. The decelerator embodiment shown in FIG. 1 can be incorporated in the vacuum housing 20 and the final energy electrode 62 is isolated from the housing 20 with insulator 66. In the presence of the decelerating electric field, space charge neutralizing electrons are swept out of the beam. The resulting diverging space charge forces are counteracted by applying a voltage (V_{f}) 65 to intermediate focusing electrode 61 via a feed-through 63 mounted on the vacuum housing 20. The voltage V_{f} is typically 0-30 kV negative with respect to electrode 62.

The embodiments for the ion decelerator are not limited to the specific arrangement shown in FIGS. 1 and 3, and one of ordinary skill in the art can appreciate a variety of implementations to optimize the ion deceleration for particular incident ion beam conditions, including: any number of workable electrodes (for example two, three, four, etc.); electrodes with circular or slot-shaped apertures; planar or curved electrodes, light or heavy non-ferromagnetic materials such as aluminum, graphite, or molybdenum for constructing the electrodes; and various vacuum configurations wherein the electrodes are installed within the magnet vacuum housing 20 or in a separate vacuum housing depending on the particular configuration of the ion implanter.

After emerging from the final energy electrode 62 the beam is transported through a beam-line 76 under vacuum to the wafer process chamber 72 to irradiate wafer 70. The wafers are processed serially one at a time, or several at a time by repeated mechanical passage of a batch wafers through the beam. Wafer 72 is admitted from and withdrawn to a clean room area via appropriate electromechanical mechanisms, doors and vacuum locks.

The embodiments of the beam-line and process chamber are not limited to a particular configuration. For example, as one of ordinary skill will appreciate, the beam-line may be simply a ballistic drift region, or it may have a number of other features including: ion optical focusing elements to provide an optimum beam size at wafer 72; beam monitoring devices; and electric or magnetic elements to sweep the beam back and forth across the wafer in order to achieve high wafer throughput with uniform irradiation dose and angular precision. The process chamber may include mechanical elements that move, the wafer relative to a beam in one or two coordinates to distribute the beam on the target. The target may have other forms from that of a circular wafer, for example it may be a rectangular substrate used in production of flat panel displays.

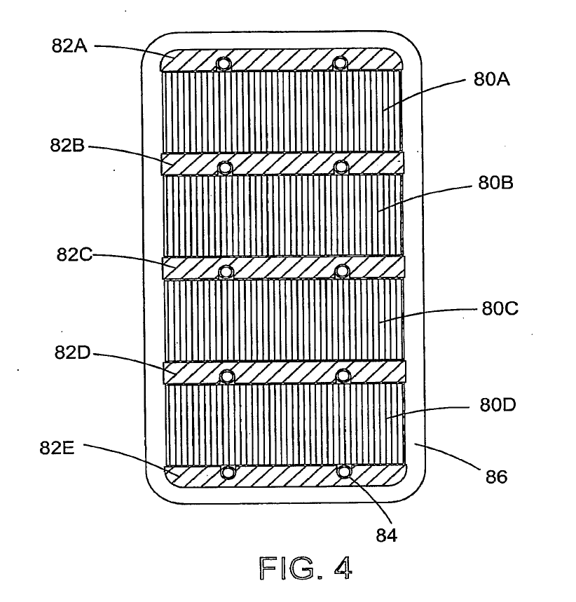

Referring to FIGS. 1 and 2 the pair of coil assemblies 40 is contoured to closely encircle and follow the general plan view shape of the poles 26 and cores 28 in order to minimize the stray magnetic flux outside the working gap between the poles and accordingly minimize the weight and cost of the yoke pieces 30, 32, and 34. In one useful commercial embodiment shown in FIG. 4, coil assembly 40 can include four separate winding elements 80A, 80B, 80C, and 80D, electrically connected in series. Winding elements 80A-D can be, for example, made of 60 turns each of copper strip 1.626 mm×38.1 mm in dimension, and wound continuously with 0.08 mm thick inter-turn electrical insulation. Insulation such as mylar or kapton are suitable. The coil current can be up to 240 A at 120V dc i.e. 28.8 kVA. This is sufficient to generate a magnetic field 24 of greater than 10 kilo-Gauss for a gap dimension of 120 mm between the poles 26.

In one embodiment, three cooling plates 82B, 82C, and 82D are disposed between respective pairs of adjacently positioned winding elements 80A-D. Outer cooling plates 82A and 82E are positioned on the outer surfaces of winding elements 80A and 80D. Cooling plates 82A-E of conductive non-ferromagnetic material such as aluminum can have any suitable thickness, for example, 10 mm. Cooling plates 82A-E provide a means for removing or dissipating ohmic heat generated from the electric current passing through winding elements 80A-D. A cooling fluid such as water can be circulated through cooling plates 82A-E via cooling tubes 84, e.g. copper tubes inserted in cooling plates 82A-E. An important aspect of the described structural embodiment is the electrical isolation of cooling tubes 84 from winding elements 80A-D. In the case of water cooling, electrical isolation of cooling tubes 84 from winding elements 80A-D significantly eliminates electrolysis and the need for using de-ionized cooling water—which, in effect, minimizes operating cost and maintenance.

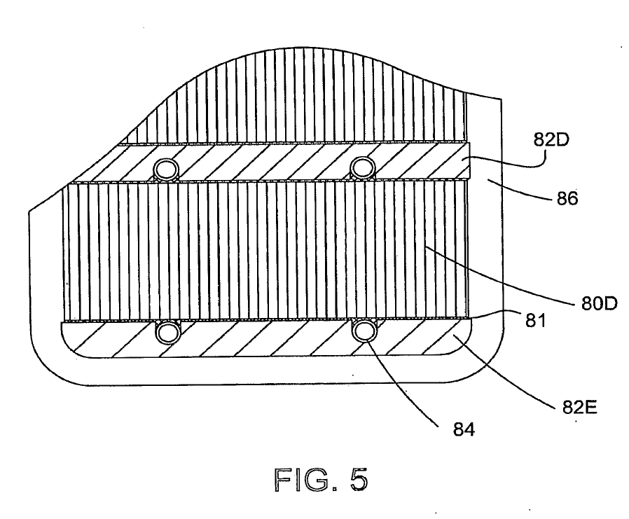

Referring to FIG. 5, in one embodiment, interleaved fiberglass cloth 81 can be used as one means for electrically isolating winding elements 80A-D from cooling plates 82A-E. The entire coil assembly 40 can also be wrapped with fiberglass tape and vacuum impregnated with epoxy resin, to effectuate a single, rigid, impervious coil assembly 40. Coil assembly 40 should possess high integrity against stress generated from thermal expansion and contraction during operation. The resin impregnated fiberglass between the edges of the winding elements 80A-D and the adjacent surfaces of cooling plates 82A-E provide high enough thermal conductivity for efficient transfer of heat which can be 29 kW in one embodiment.

The embodiment of the coil assembly should not be limited to the aforementioned description. One of ordinary skill in the art can appreciate a variety of implementations, including: any workable number of windings and cooling plates (for example two, and three, respectively); other suitable materials used for winding elements such as aluminum. Additionally, winding elements can be made by using rectangular, square, or solid copper or aluminum wire rather than strip. In an alternative embodiment, rectangular, square, or circular copper or aluminum tube can be used for the winding elements which can be directly cooled by passing a de-ionized cooling fluid through the hole of the conductor tube, rather than using indirect cooling by thermal conduction to cooling plates.

Inter-turn insulation can be implemented by other methods and materials, such as wrapping the conductor with an insulating tape, sliding an insulating sleeve over the conductor, or coating the conductor with an insulating film, e.g. enameled copper or anodized aluminum.

When the ion decelerator is activated, the magnet vacuum housing 20, and other parts of the magnet electrically connected to the vacuum housing, such as the poles 26, cores 28, and yoke parts 30, 32, and 34, all must become electrically biased from ground potential by a voltage corresponding to the decelerating voltage V_{d} (64), i.e. by a voltage in the range of 0-30 kV negative with respect to ground potential.

In one important aspect of the embodiment, the integral windings 80A-D and cooling plates 82A-E are wrapped in porous insulating material such as fiber glass and vacuum impregnated with epoxy to form an impervious cocoon 86 around the entire coil assembly 40 approximately 6-8 mm in thickness, to serve as a high voltage insulator. In another embodiment an insulating powder such as aluminum oxide can be used instead of fiberglass to fill the epoxy, and the cocoon formed using a casting mold. The high voltage insulating cocoon 86 enables the coil assembly to be electrically isolated by up to a voltage of 30 kV from the remainder of the magnet structure, namely the cores 28, poles 26, vacuum housing 20, and yoke pieces 30, 32, and 34. Therefore, the windings 80A-D and the cooling plates 82A-E can remain nominally at ground potential even though the remainder of the magnet may have up to 30 kV negative bias with respect to ground potential—which, in effect, provides a substantial cost benefit because the coil power supplies 100 can be operated at ground potential using standard grounded ac power 102. The embodiment described avoids the need to provide isolation of the coil power supplies 100 to 30 kV. More importantly, it also avoids the need to use a 30 kV isolation transformer for the 30-40 kVA input ac power for the coil power supplies 100. A further advantage lies in the fact that the fluid cooling needed to remove the heat collected in cooling plates 82A-E, for example 29 kW in one embodiment, can be provided from a ground potential source 98 without the need to use a de-ionized fluid. In fact the cooling fluid can be regular non-de-ionized tap water.

Referring to FIGS. 1 and 2, the current terminals 87 for the windings penetrate the high voltage insulating cocoon 86 at a location that is typically a distance of 40 mm or greater from any neighboring components of the magnet to enable up to 30 kV electrical isolation to be applied to the coil windings 80A-D and cooling plates 82A-E without arcing and electrical breakdown occurring between the coil terminals 87 and the magnet surround. Similarly, the cooling tubes 88 are brought out through the cocoon 86 in a manner that provides a safe working distance of at least 40 mm from the magnet surround, again to avoid arcing and electrical breakdown. The cooling tubes are welded into manifold 89 which is constructed with radii on its edges and corners in order to eliminate electrical coronas. It is also positioned to avoid arcing and electrical breakdown to the magnet surround.

The embodiments for forming the high voltage insulator around the coil assembly and bringing winding terminals and cooling tubes outside the coil should not be limited to the aforementioned method. One of ordinary skill in the art can appreciate a variety of implementations including using a powder.

The current leads 90 and cooling lines 92 pass from the coil to a ground surround 96 via insulating PVC sleeves 94 passing through the magnet yoke return 32.

What is claimed is:

1. A magnetic analysis apparatus for use with a decelerator for post analysis deceleration of ions for ion implantation, the apparatus comprising a sector magnet (21) associated with a vacuum housing (20) of nonmagnetic material through which an ion beam passes, the sector magnet having a magnet assembly, (26, 28, 30, 32, 34) of ferromagnetic material defining a magnetic field gap to which the ion beam (19, 22) is exposed for mass separation and an excitation coil (40) closely associated with the magnet assembly, the coil connected to power leads (90) extending to a power supply (100) and cooling fluid lines (92) extending to a cooling fluid source and drain, wherein high voltage insulation (86, 94) isolates the closely associated excitation coil (40), power leads and cooling fluid lines from the magnet assembly and the power supply is disposed in a grounded housing (96).

2. The apparatus of claim 1 in which the analyzer magnet (21) and its power supply (100) are constructed to operate with power of at least 20 kilowatts.

3. The apparatus of claim 1 in which at least one sleeve (94) forming a high voltage insulator extends through a portion of the magnet assembly to the excitation coil (40), the sleeve containing the electrical power leads (90) and cooling fluid lines (92).

4. The apparatus of claim 1 in which the excitation coil (40) is surrounded by electrical insulation (86) capable of providing electrical isolation from the magnet assembly (21) of least 20 kV.

5. The apparatus of claim 1 any of the foregoing claim in which the excitation coil (40) comprises an assembly of alternating coil segments (80A, B, C, D) and cooling plates (82 A, B, C, D, E) having coolant passages, the excitation coil connected to the power leads (90) and the cooling plates connected to the cooling fluid lines (92), and a high voltage insulator layer (86) encapsulates the assembly.

6. The apparatus of claim 5 in which the high voltage insulator layer (86) is in the form of an impervious cocoon of insulating material of at least 6 mm thickness.

7. The apparatus of claim 1 associated with a vacuum housing (20) held at the same voltage potential as the magnet assembly (21), the magnet assembly comprising yoke (30, 32, 34) and core (28) members disposed outside of the housing and pole members (26) that extend through and are sealed to walls of the vacuum housing (20), faces of the pole members at the inside of the housing defining the gap for the ion beam (22) and surfaces of the pole members at the outside of the housing defining flux interfaces removably related to matching surfaces of the core members (28) of the magnet assembly.

8. The apparatus of claim 1 in which the vacuum housing for the mass analyzer has a housing extension in which an ion decelerator (60, 61, 62) is mounted, the housing extension constructed to be held at the same voltage potential as the housing (20) for the mass analyzer.

9. The apparatus of claim 8 in which the decelerator comprises an assembly that includes a final energy electrode (62), the final energy electrode supported from the housing for the mass analyzer by a high voltage insulator (66).

10. The apparatus of claim 1 in which the mass analyzer is enclosed in a high voltage enclosure (E) that is isolated by high voltage insulators from electrical ground, and the power supply (100) for the excitation coil (40) is outside of the high voltage enclosure.

11. The apparatus of claim 1 in which the cooling fluid supply line (92) is connected to a source of water (98) that is not de-ionized.

12. The apparatus of claim 1 in which the sector magnet (21) extends over an arc of about 120 degrees and defines a gap of at least 100 mm dimension.

13. A method of conducting ion implantation implemented by use of the apparatus of claim 1.
